(19) United States
(12) Patent Application Publication
(10) Pub. No.: US 2008/0090873 A1
(43) Pub. Date: Apr. 17, 2008

(54) Sulfonamide derivatives

(76) Inventors: Lyn Howard Jones, Sandwich (GB); Graham Lunn, Sandwich (GB); David Anthony Price, Groton, CT (US)

Correspondence Address: PFIZER INC., PATENT DEPARTMENT, MS8260-1611, EASTERN POINT ROAD, GROTON, CT, 06340, US

(21) Appl. No.: 11/906,796

(22) Filed: Oct. 2, 2007

Related U.S. Application Data
(60) Provisional application No. 60/828,099, filed on Oct. 4, 2006.

(51) Int. Cl. A61K 31/4465; A61P 11/00; C07D 211/46

(52) U.S. Cl. 514/327; 546/222

(57) ABSTRACT

The invention relates to compounds of formula (1) 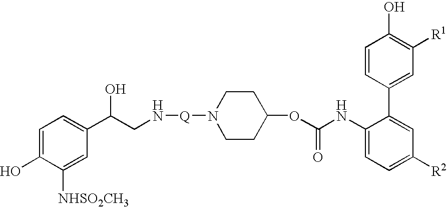 and to processes for the preparation of, intermediates used in the preparation of, compositions containing and the uses of, such derivatives. The compounds according to the present invention are useful in numerous diseases, disorders and conditions, in particular inflammatory, allergic and respiratory diseases, disorders and conditions.

This invention relates to compounds of general formula (1): 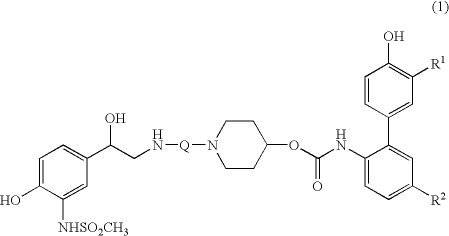
in which R¹, R² and Q have the meanings indicated below, and to processes and intermediates for the preparation of, compositions containing and the uses of such derivatives.

B₂ adrenergic agonists and cholinergic muscarinic antagonists are well-established therapeutic agents for the treatment of obstructive respiratory diseases such as COPD and asthma. Currently used inhaled β₂ agonists include both short acting agents such as salbutamol (q.i.d.), and terbutaline (t.i.d) and longer acting agents such as salmeterol, and formoterol (b.i.d.) and produce bronchodilation via stimulation of adrenergic receptors on airway smooth muscle. Inhaled Muscarinic antagonists in clinical use include the short acting ipratropium bromide (q.i.d.), oxitropium bromide (q.i.d) and the long acting tiotropium (q.d.). Muscarinic antagonists produce bronchodilation by inhibiting the cholinergic tone of airways primarily by antagonising the action of acetylcholine on muscarinic receptors present on airway smooth muscle. A number of published studies have demonstrated that the combined administration of inhaled β₂ agonists with inhaled muscarinic antagonists (whether short or long acting) to patients with obstructive lung disease results in superior improvements in lung function, symptoms and quality of life measures compared to patients receiving either single class of agent alone. Studies to date have been restricted to combination studies with single pharmacology agents, however combination of both pharmacologies within a single molecule would be desirable as this could yield increased bronchodilator efficacy with similar therapeutic index to the single agents or similar efficacy with superior therapeutic index. In addition, combining both pharmacologies in a single molecule would allow the potential for combination with anti-inflammatory agents thus giving a triple therapy from a single inhaler. Accordingly, there is a need for novel compounds active as beta 2 agonist and M3 antagonists that would have an appropriate pharmacological profile, for example in terms of potency, selectivity, pharmacokinetics, safety, systemic exposure or duration of action. In particular, there is a need for compounds suitable for an administration by the inhalation route. In this context, the present invention relates to novel compounds active as β₂ agonists and muscarinic antagonists.

The invention relates to the compounds of general formula (1): 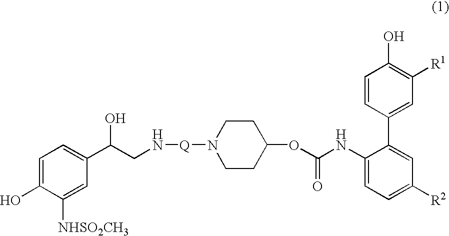
wherein
R¹ is halo,
R² is H or halo, and,
Q is selected from —(CH₂)₉— or 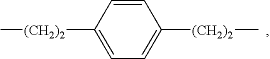
or, if appropriate, their pharmaceutically acceptable salts and/or solvates thereof.

In the here above general formula (1), halo denotes a halogen atom selected from the group consisting of fluoro, chloro, bromo and iodo in particular fluoro or chloro.

The compounds of formula (1) are β2 adrenergic receptor agonists and muscarinic receptor antagonists that are particularly useful for the treatment of diseases and/or conditions involving said receptors, by showing excellent potency, in particular when administered via the inhalation route.

The compounds of the formula (1) 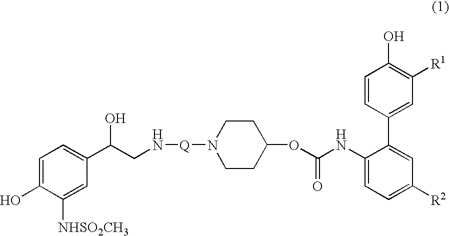
can be prepared using conventional procedures such as by the following illustrative methods in which R¹, R² and Q and are as previously defined for the compounds of the formula (1) unless otherwise stated.

The amine derivative of the formula (1) may be prepared by reaction of an amine of formula (2): 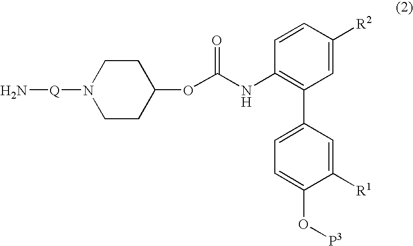
wherein R¹, R² and Q are as previously defined, with a bromide of formula (3): 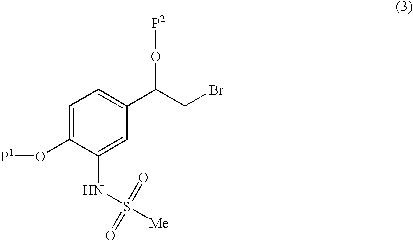
wherein P¹ and P² are suitable hydroxyl protecting groups. Preferably P¹ is benzyl and P² is TBDMS. P³ is an optional suitable hydroxyl protecting group. Preferably, P³ is benzyl.

In a typical procedure, the amine of formula (2) is reacted with a bromide of formula (3) optionally in the presence of a solvent or mixture of solvents (e.g. dimethyl sulphoxide, toluene, N,N-dimethylformamide, propionitrile, acetonitrile), optionally in the presence of a suitable base (e.g. triethylamine, diisopropylethylamine, potassium carbonate, potassium hydrogen carbonate) at a temperature comprised between 80° C. and 120° C., for 12 to 48 hours. The protecting groups can then be removed using standard methodology for cleaving oxygen protecting groups such as those found in the text book T. W. Greene, Protective Groups in Organic Synthesis, A. Wiley-Interscience Publication, 1981.

The bromide of formula (3) may be prepared according to the method of WO 2005/080324.

The amine of formula (2) may be prepared from the corresponding protected amine of formula (4): 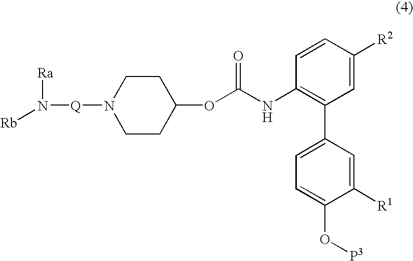
wherein Ra and Rb represent any suitable substituents so that the bonds between N and Ra and N and Rb may be easily cleaved to give the free amine of formula (2) using standard methodology for cleaving nitrogen protecting groups such as those found in the text book T. W. Greene, Protective Groups in Organic Synthesis, A. Wiley-Interscience Publication, 1981. For example Ra and Rb could be selected from allyl, benzyl, t-butyl carbamate or when joined together to form phthalimide.

The amine of formula (4) may be prepared from the corresponding amine of formula (5): 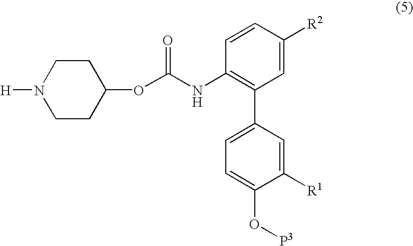
with a bromide of formula (6):

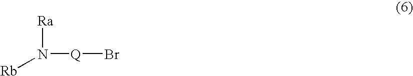

In a typical procedure, the amine of formula (5) is reacted with a bromide of formula (6) optionally in the presence of a solvent or mixture of solvents (e.g. dimethyl sulphoxide, toluene, N,N-dimethylformamide, propionitrile, acetonitrile), optionally in the presence of a suitable base (e.g. triethylamine, diisopropylethylamine, potassium carbonate, potassium hydrogen carbonate) at a temperature comprised between 80° C. and 120° C., for 12 to 48 hours.

The bromide of formula (6) may be prepared from the corresponding dibromide of formula (7) and the corresponding amine nucleophile RaRbNH wherein Ra and Rb represent any suitable substituents so that the bonds between N and Ra and Rb may be easily cleaved.
Br-Q-Br (7)

In a typical procedure the bromide (7) is reacted with the sodium salt of phthalimide or di-tert-butyl iminodicarbonate in a solvent such as dimethyl sulfoxide, toluene, N,N-dimethylformamide, acetonitrile, tetrahydrofuran at a temperature comprised between 0° C. and 150° C. for 6-48 hours. The dibromide of formula (7) where Q is —(CH₂)₉— is commercially available.

The dibromide of formula (7) where Q is 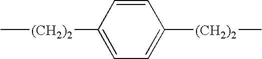
may be prepared from the corresponding diol of formula (8):

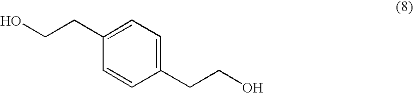

In a typical procedure the diol (8) is treated with a suitable brominating reagent such as PBr₃ or HBr optionally in the presence of a solvent (eg chloroform, dichloromethane, tetrahydrofuran) at a temperature between comprised between 0° C. and 150° C. for 6-48 hours.

The diol (8) may be prepared from the commercially available diacid (9):

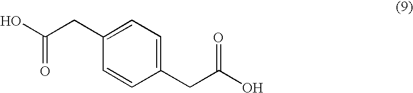

In a typical procedure the diacid (9) is treated with a suitable reducing reagent such as lithium aluminium hydride or borane in the presence of a solvent (eg chloroform, dichloromethane, tetrahydrofuran, diethyl ether) at a temperature between comprised between −78° C. and 150° C. for 1-48 hours.

The amine (5) may be prepared from the bromide of formula (10) and the commercially available aryl boronic acid.

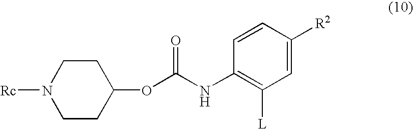

Rc is selected so that it may be easily cleaved to give the free amine of formula (5). L is a leaving group, preferably bromo or iodo.

In a typical procedure, the aryl halide of formula (10) is reacted with aryl boronic acid in the presence of a suitable palladium catalyst (palladium acetate/tri-ortho-tolylphosphine of formula Pd(OAc)₂/P(o-Tol)₃) in a solvent (e.g. toluene, benzene, hexane, dimethoxyethane, N,Ndimethylformamide) in the presence of a base (e.g. sodium hydrogencarbonate, casium carbonate, triethylamine). Preferably, the reaction is carried out at a temperature comprised between 80° C. and 110° C. for 4 to 16 hours. Rc is then cleaved using standard methodology for cleaving nitrogen protecting groups such as those found in the text book T. W. Greene, Protective Groups in Organic Synthesis, A. Wiley-Interscience Publication, 1981.

Alternatively the amine of formula (4) may be prepared from the corresponding protected amine of formula (11) and the commercially available boronic acid.

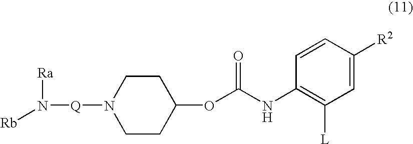

In a typical procedure, the aryl halide of formula (11) is reacted with aryl boronic acid in the presence of a suitable palladium catalyst (palladium acetate/tri-ortho-tolylphosphine of formula Pd(OAc)₂/P(o-Tol)₃) in a solvent (e.g. toluene, benzene, hexane, dimethoxyethane, N,N-dimethylformamide) in the presence of a base (e.g. sodium hydrogencarbonate, caesiumcarbonate, triethylamine). Preferably, the reaction is carried out at a temperature comprised between 80° C. and 110° C. for 4 to 16 hours.

Alternatively the compound of formula (1) can be prepared from the corresponding bromide of formula (12) and the commercially available boronic acid.

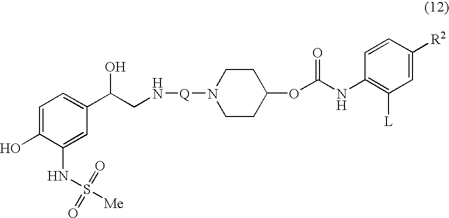

In a typical procedure, the aryl halide of formula (12) is reacted with aryl boronic acid in the presence of a suitable palladium catalyst (palladium acetate/tri-ortho-tolylphosphine of formula Pd(OAc)₂/P(o-Tol)₃) in a solvent (e.g. toluene, benzene, hexane, dimethoxyethane, N,N-dimethylformamide) in the presence of a base (e.g. sodium hydrogencarbonate, casium carbonate). Preferably, the reaction is carried out at a temperature comprised between 80° C. and 110° C. for 4 to 16 hours. Optionally the hydroxyls and basic centre may be protected using standard methodology such as those found in the text book T. W. Greene, Protective Groups in Organic Synthesis, A. Wiley-Interscience Publication, 1981.

The bromide of formula (12) may be prepared from the corresponding protected compound of formula (13): 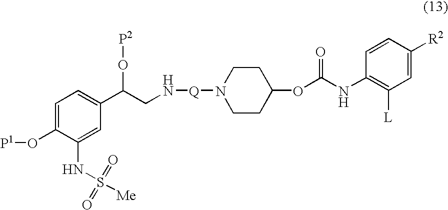
wherein P¹ and P² are suitable hydroxyl protecting groups. Preferably P¹ is benzyl and P² is TBDMS.

The protecting groups may be easily cleaved to give the bromide of formula (12) using standard methodology for cleaving hydroxy protecting groups such as those found in the text book T. W. Greene, Protective Groups in Organic Synthesis, A. Wiley-Interscience Publication, 1981.

The bromide of formula (13) may be prepared from the bromide of formula (3) and the deprotected amine of formula (11) where Ra and Rb=H. In a typical procedure, the amine of formula (11) is reacted with a bromide of formula (3) optionally in the presence of a solvent or mixture of solvents (e.g. dimethyl sulphoxide, toluene, N,N-dimethylformamide, propionitrile, acetonitrile), optionally in the presence of a suitable base (e.g. triethylamine, diisopropylethylamine, potassium carbonate, potassium hydrogen carbonate) at a temperature comprised between 80° C. and 120° C., for 12 to 48 hours.

The amine of formula (11) may be prepared from the corresponding amine of formula (14): 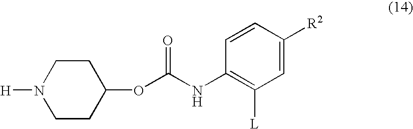
with a bromide of formula (6):

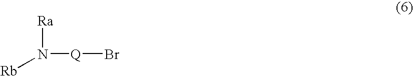

In a typical procedure, the amine of formula (14) is reacted with a bromide of formula (6) optionally in the presence of a solvent or mixture of solvents (e.g. dimethyl sulphoxide, toluene, N,N-dimethylformamide, propionitrile, acetonitrile), optionally in the presence of a suitable base (e.g. triethylamine, diisopropylethylamine, potassium carbonate, potassium hydrogen carbonate) at a temperature comprised between 80° C. and 120° C., for 12 to 48 hours.

The amine of formula (14) may be prepared from the corresponding protected amine of formula (15) and the corresponding isocyanate.

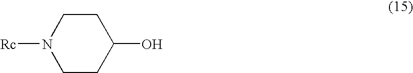

The isocyanate can be commercial or prepared as an intermediate from the corresponding amine or carboxylic acid. In a typical procedure the amine (15) is treated with the isocyanate optionally in the presence of a solvent or mixture of solvents (e.g. dimethyl sulphoxide, toluene, N,N-dimethylformamide, acetonitrile, tetrahydrofuran), optionally in the presence of a suitable base (e.g. triethylamine, diisopropylethylamine, potassium carbonate, potassium hydrogen carbonate) at a temperature comprised between 0° C. and 80° C., for 1 to 48 hours. Rc is selected so that it may be easily cleaved to give the free amine of formula (5) using standard methodology for cleaving nitrogen protecting groups such as those found in the text book T. W. Greene, Protective Groups in Organic Synthesis, A. Wiley-Interscience Publication, 1981.

For some of the steps of the here above described process of preparation of the compounds of formula (1), it may be necessary to protect potential reactive functions that are not wished to react, and to cleave said protecting groups in consequence. In such a case, any compatible protecting radical can be used. In particular methods of protection and deprotection such as those described by T. W. GREENE (Protective Groups in Organic Synthesis, A. Wiley-Interscience Publication, 1981) or by P. J. Kocienski (Protecting groups, Georg Thieme Verlag, 1994), can be used.

All of the above reactions and the preparations of novel starting materials used in the preceding methods are conventional and appropriate reagents and reaction conditions for their performance or preparation as well as procedures for isolating the desired products will be well-known to those skilled in the art with reference to literature precedents and the examples and preparations hereto.

Also, the compounds of formula (1) as well as intermediate for the preparation thereof can be purified according to various well-known methods, such as for example crystallization or chromatography.

Subgroups of compounds of formula (1) containing the following substituents or combination of substituents are preferred:
R¹ is F or Cl, and/or,
R² is H, F or Cl, preferably H or F, and/or
Q is —(CH₂)₉—.

Particularly preferred, compounds according to the invention are:
1-(9-{[(2R)-2-Hydroxy-2-{4-hydroxy-3-[(methylsulfonyl)amino]phenyl}ethyl]amino}nonyl)piperidin-4-yl(3′-fluoro-4′-hydroxybiphenyl-2-yl)carbamate,
1-(9-{[(2R)-2-Hydroxy-2-{4-hydroxy-3-[(methylsulfonyl)amino]phenyl}ethyl]amino}nonyl)piperidin-4-yl(3′-chloro-4′-hydroxybiphenyl-2-yl)carbamate, and,
1-(9-{[(2R)-2-hydroxy-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate,
   or, if appropriate, their pharmaceutically acceptable salts and/or solvates thereof.

Pharmaceutically acceptable salts of the compounds of formula (1) include the acid addition and base salts thereof.

Suitable acid addition salts are formed from acids which form non-toxic salts. Examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of formula (1) may be prepared by one or more of three methods:
(i) by reacting the compound of formula (1) with the desired acid or base;
(ii) by removing an acid- or base-labile protecting group from a suitable precursor of the compound of formula (1) or by ring-opening a suitable cyclic precursor, for example, a lactone or lactam, using the desired acid or base; or
(iii) by converting one salt of the compound of formula (1) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

Hereinafter all references to compounds of formula (1) include references to salts, solvates and complexes thereof and to solvates and complexes of salts thereof.

The compounds of the invention include compounds of formula (1) as hereinbefore defined, including all polymorphs and crystal habits thereof, prodrugs and isomers thereof (including optical, geometric and tautomeric isomers) as hereinafter defined and isotopically-labeled compounds of formula (1).

As indicated, so-called ‘pro-drugs’ of the compounds of formula (1) are also within the scope of the invention. Thus certain derivatives of compounds of formula (1) which may have little or no pharmacological activity themselves can, when administered into or onto the body, be converted into compounds of formula (1) having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T. Higuchi and W. Stella) and ‘Bioreversible Carriers in Drug Design’, Pergamon Press, 1987 (ed. E. B Roche, American Pharmaceutical Association).

Prodrugs in accordance with the invention can, for example, be produced by replacing appropriate functionalities present in the compounds of formula (1) with certain moieties known to those skilled in the art as ‘pro-moieties’ as described, for example, in “Design of Prodrugs” by H. Bundgaard (Elsevier, 1985).

Some examples of prodrugs in accordance with the invention include:

(i) where the compound of formula (1) contains an alcohol functionality (—OH), an ether thereof, for example, a compound wherein the hydrogen of the alcohol functionality of the compound of formula (1) is replaced by (C₁-C₆)alkanoyloxymethyl; and

(ii) where the compound of formula (1) contains a primary or secondary amino functionality (—NH₂ or —NHR where R≠H), an amide thereof, for example, a compound wherein, as the case may be, one or both hydrogens of the amino functionality of the compound of formula (1) is/are replaced by (C₁-C₁₀)alkanoyl.

Further examples of replacement groups in accordance with the foregoing examples and examples of other prodrug types may be found in the aforementioned references.

Moreover, certain compounds of formula (1) may themselves act as prodrugs of other compounds of formula (1).

Also included within the scope of the invention are metabolites of compounds of formula (1), that is, compounds formed in vivo upon administration of the drug. Some examples of metabolites in accordance with the invention include

(i) where the compound of formula (1) contains a secondary amino group, a primary derivative thereof (—NHR¹→—NH₂), and

(ii) where the compound of formula (1) contains a phenyl moiety, a phenol derivative thereof (-Ph→-PhOH).

Included within the scope of the present invention are all stereoisomers, geometric isomers and tautomeric forms of the compounds of formula (1), including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula (1) contains an acidic or basic moiety, an acid or base such as tartaric acid or 1-phenylethylamine. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

Stereoisomeric conglomerates may be separated by conventional techniques known to those skilled in the art—see, for example, “Stereochemistry of Organic Compounds” by E. L. Eliel (Wiley, New York, 1994).

According to one aspect of the present invention, the (R)-stereoisomer of the formula below, wherein R¹, R² and Q are as defined in claim 1 is preferred:

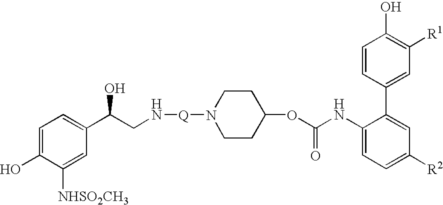

The present invention includes all pharmaceutically acceptable isotopically-labelled compounds of formula (1) wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula (1), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula (1) can generally be prepared by conventional techniques known to those skilled in the art or by processes analogous to those described in the accompanying Examples and Preparations using an appropriate isotopically-labeled reagents in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The compounds of formula (1), their pharmaceutically acceptable salts and/or derived forms, are valuable pharmaceutically active compounds, which are suitable for the therapy and prophylaxis of numerous disorders in which agonism of the β2 receptor and antagonism of the muscarinic receptor may induce benefit, in particular the allergic and non-allergic airways diseases.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term “excipient” is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in ‘Remington's Pharmaceutical Sciences’, 19th Edition (Mack Publishing Company, 1995).

The compounds of the invention may be administered orally. Oral administration may involve swallowing, so that the compound enters the gastrointestinal tract, or buccal or sublingual administration may be employed by which the compound enters the blood stream directly from the mouth.

Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (1), a film-forming polymer, a binder, a solvent, a humectant, a plasticiser, a stabiliser or emulsifier, a viscosity-modifying agent and a solvent. Some components of the formulation may perform more than one function.

The compound of formula (1) may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula (1) may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula (1) used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a solid, semi-solid, or thixotropic liquid for administration as an implanted depot providing modified release of the active compound. Examples of such formulations include drug-coated stents and poly(dl-lactic-coglycolic)acid (PGLA) microspheres.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958 by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (1), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from 0.001 mg to 10 mg of the compound of formula (1). The overall daily dose will typically be in the range 0.001 mg to 40 mg which may be administered in a single dose or, more usually, as divided doses throughout the day.

The compounds of formula (1) are particularly suitable for an administration by inhalation

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis.

Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula (1) in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example parenteral, for administering the separate compositions at different dosage intervals, or for titrating the separate compositions against one another. To assist compliance, the kit typically comprises directions for administration and may be provided with a so-called memory aid.

For administration to human patients, the total daily dose of the compounds of the invention is typically in the range 0.001 mg to 5000 mg depending, of course, on the mode of administration. For example, an intravenous daily dose may only require from 0.001 mg to 40 mg. The total daily dose may be administered in single or divided doses and may, at the physician's discretion, fall outside of the typical range given herein.

These dosages are based on an average human subject having a weight of about 65 kg to 70 kg. The physician will readily be able to determine doses for subjects whose weight falls outside this range, such as infants and the elderly.

For the avoidance of doubt, references herein to “treatment” include references to curative, palliative and prophylactic treatment.

According to another embodiment of the present invention, the compounds of the formula (1), or pharmaceutically acceptable salts, derived forms or compositions thereof, can also be used as a combination with one or more additional therapeutic agents to be co-administered to a patient to obtain some particularly desired therapeutic end result such as the treatment of pathophysiologically-relevant disease processes including, but not limited to (i) bronchoconstriction, (ii) inflammation, (iii) allergy, (iv) tissue destruction, (v) signs and symptoms such as breathlessness, cough. The second and more additional therapeutic agents may also be a compound of the formula (1), or a pharmaceutically acceptable salt, derived forms or compositions thereof, or one or more β2 agonists, muscarinic antagonists or compounds active as beta 2 agonist and as muscarinic antagonist known in the art. More typically, the second and more therapeutic agents will be selected from a different class of therapeutic agents.

As used herein, the terms “co-administration”, “co-administered” and “in combination with”, referring to the compounds of formula (1) and one or more other therapeutic agents, is intended to mean, and does refer to and include the following:
simultaneous administration of such combination of compound(s) of formula (1) and therapeutic agent(s) to a patient in need of treatment, when such components are formulated together into a single dosage form which releases said components at substantially the same time to said patient,
substantially simultaneous administration of such combination of compound(s) of formula (1) and therapeutic agent(s) to a patient in need of treatment, when such components are formulated apart from each other into separate dosage forms which are taken at substantially the same time by said patient, whereupon said components are released at substantially the same time to said patient,
sequential administration of such combination compound(s) of formula (1) and therapeutic agent(s) to a patient in need of treatment, when such components are formulated apart from each other into separate dosage forms which are taken at consecutive times by said patient with a significant time interval between each administration, whereupon said components are released at substantially different times to said patient; and
sequential administration of such combination of compound(s) of formula (1) and therapeutic agent(s) to a patient in need of treatment, when such components are formulated together into a single dosage form which releases said components in a controlled manner whereupon they are concurrently, consecutively, and/or overlapingly administered at the same and/or different times by said patient,
   where each part may be administered by either the same or different route.

Suitable examples of other therapeutic agents which may be used in combination with the compound(s) of formula (1), or pharmaceutically acceptable salts, derived forms or compositions thereof, include, but are by no means limited to:
(a) 5-Lipoxygenase (5-LO) inhibitors or 5-lipoxygenase activating protein (FLAP) antagonists,
(b) Leukotriene antagonists (LTRAs) including antagonists of LTB₄, LTC₄, LTD₄, and LTE₄,
(c) Histamine receptor antagonists including H1 and H3 antagonists,
(d) α₁- and α₂-adrenoceptor agonist vasoconstrictor sympathomimetic agents for decongestant use,
(e) PDE inhibitors, e.g. PDE3, PDE4 and PDE5 inhibitors,
(f) Theophylline,
(g) Sodium cromoglycate,
(h) COX inhibitors both non-selective and selective COX-1 or COX-2 inhibitors (NSAIDs),
(i) Prostaglandin receptor antagonists and inhibitors of prostaglandin synthase,
(j) Oral and inhaled glucocorticosteroids,
(k) Monoclonal antibodies active against endogenous inflammatory entities,
(l) Anti-tumor necrosis factor (anti-TNF-α) agents,
(m) Adhesion molecule inhibitors including VLA-4 antagonists,
(n) Kinin-B₁- and B₂-receptor antagonists,
(o) Immunosuppressive agents,
(p) Inhibitors of matrix metalloproteases (MMPs),
(q) Tachykinin NK₁, NK₂ and NK₃ receptor antagonists,
(r) Elastase inhibitors,
(s) Adenosine A2a receptor agonists,
(t) Inhibitors of urokinase,
(u) Compounds that act on dopamine receptors, e.g. D2 agonists,
(v) Modulators of the NFκβ pathway, e.g. IKK inhibitors,
(w) modulators of cytokine signalling pathyways such as p38 MAP kinase, syk kinase, or JAK kinase inhibitors,
(x) Agents that can be classed as mucolytics or anti-tussive,
(y) Agents which enhance responses to inhaled corticosteroids,
(z) Antibiotics and antiviral agents effective against micro-organisms which can colonise the respiratory tract,
(aa) Prostaglandin antagonists such as DP1, DP2 or CRTH2 antagonists,
(bb) HDAC inhibitors,
(cc) PI3 kinase inhibitors,
(dd) p38 inhibitors, and,
(ee) CXCR2 antagonists.

According to the present invention, combination of the compounds of formula (1) with

H3 antagonists,

PDE4 inhibitors,

glucocorticosteroids,

Adenosine A2a receptor agonists,

Modulators of cytokine signalling pathyways such as p38 MAP kinase or syk kinase, or,

Leukotriene antagonists (LTRAs) including antagonists of LTB₄, LTC₄, LTD₄, and LTE₄, are preferred.

According to the present invention, combination of the compounds of formula (1) with
glucocorticosteroids, in particular inhaled glucocorticosteroids with reduced systemic side effects, including prednisone, prednisolone, flunisolide, triamcinolone acetonide, beclomethasone dipropionate, budesonide, fluticasone propionate, ciclesonide, and mometasone furoate,
   are further preferred.

It is to be appreciated that all references herein to treatment include curative, palliative and prophylactic treatment.

The compounds of formula (1) have the ability to interact with the β₂ receptor and cholinergic muscarinic receptors, and thereby have a wide range of therapeutic applications, as described further below, because of the essential role which the β2 receptor and muscarinic receptors play in the physiology of all mammals.

Therefore, a further aspect of the present invention relates to the compounds of formula (1), or pharmaceutically acceptable salts, derived forms or compositions thereof, for use in the treatment of diseases, disorders, and conditions in which the β2 receptor and/or muscarinic receptors are involved. More specifically, the present invention also concerns the compounds of formula (1), or pharmaceutically acceptable salts, derived forms or compositions thereof, for use in the treatment of diseases, disorders, and conditions selected from the group consisting of:
asthma of whatever type, etiology, or pathogenesis, in particular asthma that is a member selected from the group consisting of atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, non-atopic asthma, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis,
chronic or acute bronchoconstriction, chronic bronchitis, small airways obstruction, and emphysema,
obstructive or inflammatory airways diseases of whatever type, etiology, or pathogenesis, in particular an obstructive or inflammatory airways disease that is a member selected from the group consisting of chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension,
bronchitis of whatever type, etiology, or pathogenesis, in particular bronchitis that is a member selected from the group consisting of acute bronchitis, acute laryngotracheal bronchitis, arachidic bronchitis, catarrhal bronchitis, croupus bronchitis, dry bronchitis, infectious asthmatic bronchitis, productive bronchitis, staphylococcus or streptococcal bronchitis and vesicular bronchitis,
acute lung injury,
bronchiectasis of whatever type, etiology, or pathogenesis, in particular bronchiectasis that is a member selected from the group consisting of cylindric bronchiectasis, sacculated bronchiectasis, fusiform bronchiectasis, capillary bronchiectasis, cystic bronchiectasis, dry bronchiectasis and follicular bronchiectasis.

A still further aspect of the present invention also relates to the use of the compounds of formula (1), or pharmaceutically acceptable salts, derived forms or compositions thereof, for the manufacture of a drug having a β2 agonist activity and an muscarinic antagonist activity. In particular, the present invention concerns the use of the compounds of formula (1), or pharmaceutically acceptable salts, derived forms or compositions thereof, for the manufacture of a drug for the treatment of diseases and/or conditions involving the beta 2 and muscarinic receptors, in particular the diseases and/or conditions listed above.

As a consequence, the present invention provides a particularly interesting method to treat a mammal, including a human being, with an effective amount of a compound of formula (1), or a pharmaceutically acceptable salt, derived form or composition thereof. More precisely, the present invention provides a particularly interesting method for the treatment of a disease and/or conditions involving the beta 2 and Muscarinic receptors, in a mammal, including a human being, in particular the diseases and/or conditions listed above, comprising administering said mammal with an effective amount of a compound of formula (1), its pharmaceutically acceptable salts and/or derived forms.

The following examples illustrate the preparation of the compounds of the formula (1):

### PREPARATION 1

### (9-Bromo-nonyl)-dicarbamic acid tert-butyl ester

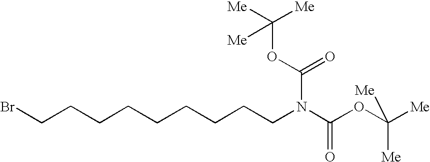

Sodium hydride (1.31 g of a 60% dispersion in oil, 30.0 mmol) was added in one portion to a stirred solution of di-tert-butyl iminodicarbamate (6.50 g, 30.0 mmol) in N,N-dimethylformamide (5 ml) at 0° C. under nitrogen. The reaction was stirred for 5 minutes at 0° C. and then stirred at room temperature for 30 minutes. The reaction was cooled to 0° C. and 1,9-dibromononane (8.60 g, 30.0 mmol) added dropwise. The reaction was allowed to warm to room temperature and stirred for 3 days. Diethyl ether (50 ml) and water (20 ml) were cautiously added and the organics separated, the aqueous layer was washed with diethyl ether (50 ml) and the combined organics dried (magnesium sulfate) and the solvent removed in vacuo to yield a clear oil. The oil was purified by column chromatography on silica gel eluting with diethyl ether:hexane (10/90 by volume) to furnish the title compound as a colourless oil, 5.80 g.

¹H NMR (400 MHz, CD₃OD): δ=1.30 (10H, m), 1.50 (20H, m), 1.83 (2H, m), 3.42 (2H, t), 3.58 (2H, t) ppm.

### PREPARATION 2

### tert-Butyl 4-({[(2-bromophenyl)amino]carbonyl}oxy)piperidine-1-carboxylate

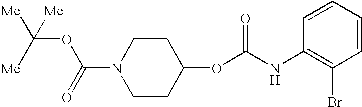

1-tert-Butoxycarbonyl-4-hydroxypiperidine (1.00 g, 5.00 mmol) was dissolved in dichloromethane (10 ml) and triethylamine (0.70 ml, 5.00 mmol) was added and the reaction stirred at room temperature for 30 minutes. A solution of 2-bromophenylisocyanate (1.00 g, 5.00 mmol) in dichloromethane (5 ml) was added dropwise over 5 minutes and the reaction left stirring at room temperature for 12 hours. The solvent was removed in vacuo to furnish an oily solid that was slurried in pentane (20 ml) for 10 minutes, the solid was filtered off to furnish the title compound as a white solid, 1.35 g.

LRMS (APCI): m/z 299 [M-boc+H]⁺.

### PREPARATION 3

### Piperidin-4-yl(2-bromophenyl)carbamate hydrochloride

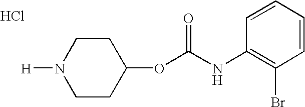

Tert-Butyl 4-({[(2-bromophenyl)amino]carbonyl}oxy)piperidine-1-carboxylate (Preparation 2, 35.0 g, 88.0 mmol) was dissolved in hydrochloric acid (175 ml of a 4M solution in dioxane) and the reaction stirred at room temperature for 30 minutes. The solvent was removed in vacuo and the resulting solid slurried in diethyl ether (100 ml) for 30 minutes. The solid was isolated by filtration to furnish the title compound as a white solid, 27.3 g.

LRMS (APCI): m/z 299 [M+H]⁺.

### PREPARATION 4

### Di-tert-Butyl {9-[4-({[(2-bromophenyl)amino]carbonyl}oxy)piperidin-1-yl]nonyl}imidodicarbonate

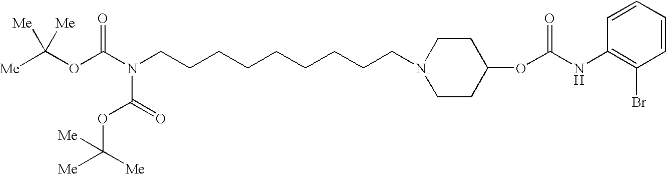

Piperidin-4-yl(2-bromophenyl)carbamate hydrochloride (Preparation 3, 4.85 g, 14.5 mmol) was suspended in acetonitrile (40 ml) and triethylamine (4.00 ml, 28.9 mmol) was added at room temperature. A solution of (9-Bromo-nonyl)-dicarbamic acid tert-butyl ester (Preparation 1, 6.10 g, 14.4 mmol) in acetonitrile (20 ml) was added dropwise and the reaction heated at 50° C. for 12 hours. The reaction was cooled to room temperature and the solvent removed in vacuo and the residue dissolved in dichloromethane (300 ml). The organics were washed with saturated aqueous sodium hydrogen carbonate (2×200 ml) and water (150 ml), dried (magnesium sulfate) and the solvent removed in vacuo to yield an oil. The oil was purified by column chromatography on silica gel eluting with pentane:ethyl acetate (50/50 by volume) to furnish the title compound, 6.50 g.

LRMS (APCI): m/z 642 [M+H]⁺.

### PREPARATION 5

### 1-(9-Aminononyl)piperidin-4-yl(2-bromophenyl)carbamate dihydrochloride

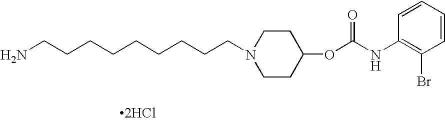

Di-tert-Butyl {9-[4-({[(2-bromophenyl)amino]carbonyl}oxy)piperidin-1-yl]nonyl}imidodicarbonate (Preparation 4, 20.0 g, 31 mmol) was dissolved in dioxane (200 ml) and hydrochloric acid (160 ml of a 4M solution in dioxane) added in one portion at room temperature. A white solid precipitated and water (50 ml) was added to dissolve the solid. The reaction was stirred at room temperature for 24 hours and the solvent removed in vacuo to furnish the tile compound as an off white solid, 18.3 g.

LRMS (APCI): m/z 441 [M+H]⁺.

### PREPARATION 6

### 1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl(2-bromophenyl)carbamate

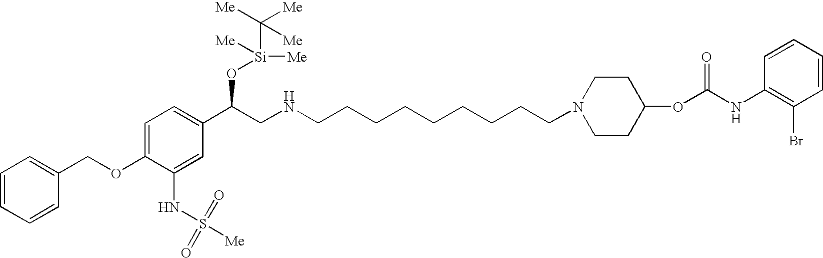

1-(9-Aminononyl)piperidin-4-yl(2-bromophenyl)carbamate dihydrochloride (Preparation 5, 16.0 g, 31.2 mmol) and N-{2-(benzyloxy)-5-[(1R)-2-bromo-1-{[tert-butyl(dimethyl)silyl]oxy}ethyl]phenyl}methanesulfonamide (WO2005/080324, 16.1 g, 31.2 mmol) and sodium hydrogencarbonate (13.1 g, 156 mmol) were heated in acetonitrile (200 ml) at 90° C. for 72 hours. The reaction was cooled to room temperature and poured onto water (20 ml) and ethyl acetate (50 ml), the organics were separated and the aqueous extracted with ethyl acetate (2×40 ml). The combined organics were dried (sodium sulfate) and the solvent removed in vacuo to furnish a brown oil. The oil was purified by column chromatography on silica gel eluting with dichloromethane:methanol:ammonia (98/2/1 by volume) to furnish the title compound as a colourless gum, 16.5 g.

LRMS (ES): m/z 877,875 [M+H]⁺.

### PREPARATION 7

### 1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl(3′-fluoro-4′-hydroxybiphenyl-2-yl)carbamate

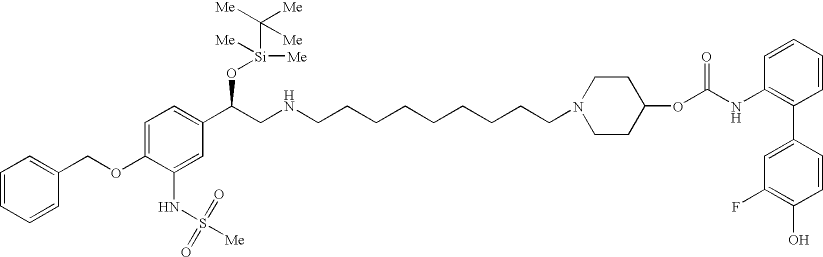

1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl(2-bromophenyl)carbamate (Preparation 6, 450 mg, 0.52 mmol), 4-hydroxy-3-fluorophenyl boronic acid (136 mg, 0.87 mmol), sodium carbonate (164 mg, 1.54 mmol), palladium acetate (7 mg, 0.03 mmol) and tri(o-tolyl)phosphine (18 mg, 0.06 mmol) were heated in dimethoxyethane (8 ml) at 80° C. under nitrogen for 12 hours. The reaction was cooled to room temperature and washed with saturated aqueous sodium hydrogen carbonate (2×30 ml), brine (30 ml), dried (magnesium sulphate) and the solvent removed in vacuo. The residue was purified by column chromatography on silica gel eluting with dichloromethane:methanol:ammonia (95/5/0.5 by volume) to furnish the title compound as a brown solid, 289 mg.

LRMS (ES): m/z 906 [M+H]⁺.

### PREPARATION 8

### 1-(9-{[(2R)-2-{[tert-Butyl(dimethyl)silyl]oxy}-2-{4-hydroxy-3-[(methylsulfonyl)amino]phenyl}ethyl]amino}nonyl)piperidin-4-yl(3′-fluoro-4′-hydroxybiphenyl-2-yl)carbamate

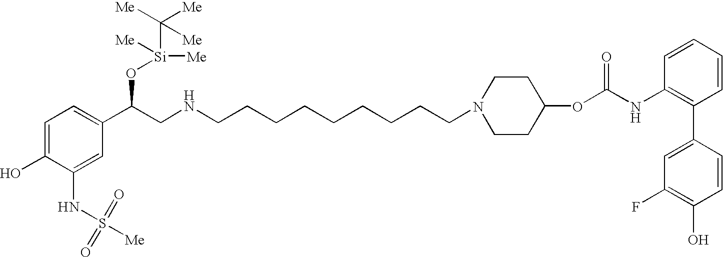

1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl(3′-fluoro-4′-hydroxybiphenyl-2-yl)carbamate (Preparation 7, 289 mg, 0.32 mmol) was dissolved in methanol (10 ml) and ammonium formate (403 mg, 6.38 mmol) and palladium hydroxide (45 mg) added in one portion. The reaction was heated under reflux for 1 hour, cooled to room temperature and further ammonium formate (100 mg) and palladium hydroxide (10 mg) added. The reaction was heated under reflux for 1 hour, cooled to room temperature and the catalyst removed by filtration through Arbocel™. The filtrate was diluted with ethyl acetate (15 ml) and washed with saturated aqueous sodium hydrogen carbonate (15 ml), brine (15 ml) and dried (magnesium sulphate). The solvent was removed in vacuo to yield the title compound as a brown oil, 264 mg.

LRMS (ES): m/z 816 [M+H]⁺.

### PREPARATION 9

### 1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl[4′-(benzyloxy)-3′-chlorobiphenyl-2-yl]carbamate

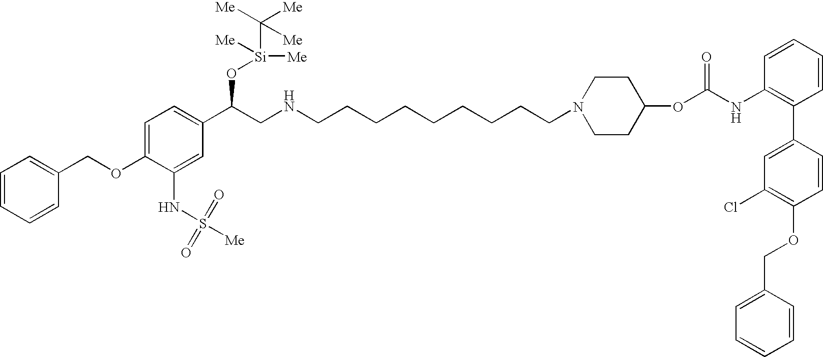

1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl(2-bromophenyl)carbamate (Preparation 6, 1000 mg, 1.14 mmol), 4-benzyloxy-3-chlorophenyl boronic acid (450 mg, 1.72 mmol), sodium carbonate (485 mg, 4.58 mmol), palladium acetate (20 mg, 0.07 mmol) and tri(o-tolyl)phosphine (42 mg, 0.14 mmol) were heated in N,N-dimethylformamide (10 ml) at 100° C. under microwave conditions for 10 minutes. The reaction was cooled to room temperature, filtered through celite and ethyl acetate (25 ml) added. The organics were washed with water (50 ml), dried (magnesium sulphate) and the solvent removed in vacuo. The residue was purified by column chromatography on silica gel eluting with dichloromethane:methanol:ammonia (95/5/0.5 by volume) to furnish the title compound as a yellow oil, 1.06 g.

LRMS (ES): m/z 1012 [M+H]⁺.

### PREPARATION 10

### 1-(9-{[(2R)-2-{[tert-Butyl(dimethyl)silyl]oxy}-2-{4-hydroxy-3-[(methylsulfonyl)amino]phenyl}ethyl]amino}nonyl)piperidin-4-yl(3′-chloro-4′-hydroxybiphenyl-2-yl)carbamate

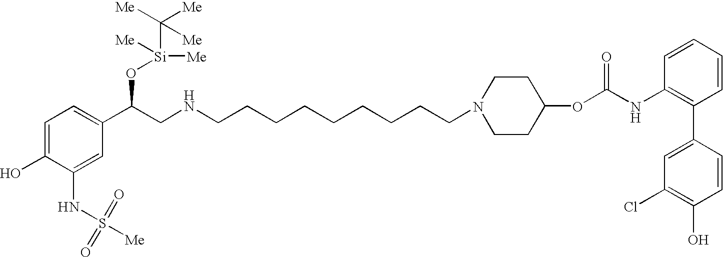

1-(9-{[(2R)-2-{4-(Benzyloxy)-3-[(methylsulfonyl)amino]phenyl}-2-{[tert-butyl(dimethyl)silyl]oxy}ethyl]amino}nonyl)piperidin-4-yl[4′-(benzyloxy)-3′-chlorobiphenyl-2-yl]carbamate (Preparation 9, 1.50 g, 1.48 mmol) was dissolved in tert-butylmethyl ether (50 ml) and 10% palladium on carbon (25 mg) added in one portion. The reaction was hydrogenated at 10 psi at room temperature for 2 hours and 1.5 hours at 15 psi. The catalyst was removed by filtration through Arbocel™ and the solvent removed in vacuo to yield the title compound as a pale yellow solid, 1.08 g.

LRMS (ES): m/z 832 [M+H]⁺.

### PREPARATION 11

### Tert-butyl 4-({[(2-bromo-4-fluorophenyl)amino]carbonyl}oxy)piperidine-1-carboxylate

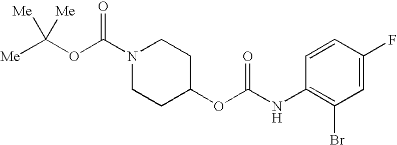

Diphenyl phosphoryl azide (1.26 g, 4.57 mmol) was added to a solution of 2-bromo-4-fluoro-benzoic acid (1 g, 4.57 mmol) and triethylamine (0.953 mL, 6.85 mmol) in toluene (80 mL) and the reaction heated to 60° C. for 10 minutes. A solution of 4-hydroxy-piperidine-1-carboxylic acid tert-butyl ester (0.919 g, 4.57 mmol) in toluene (20 mL) was added dropwise over 20 minutes. The reaction mixture was heated under nitrogen at 60° C. for 8 hours. Reaction solvent was removed in vacuo. Residue was dissolved in ethyl acetate (50 mL) and washed with water (30 mL). Organics were separated and then aqueous layer was washed with ethyl acetate (50 mL). The combined organics were dried (sodium sulfate) and concentrated in vacuo to yield a yellow coloured oil. The oil was purified by column chromatography on silica gel eluting with ethyl acetate:heptane (10/90 by volume) to ethyl acetate:heptane (30/70 by volume) to furnish the title compound as a colourless oil, 1.35 g

LRMS (ESI): m/z 317/319 [(M-BOC)H⁺

### PREPARATION 12

### tert-butyl 4-[({[4′-(benzyloxy)-3′-chloro-5-fluorobiphenyl-2-yl]amino}carbonyl)oxy]piperidine-1-carboxylate

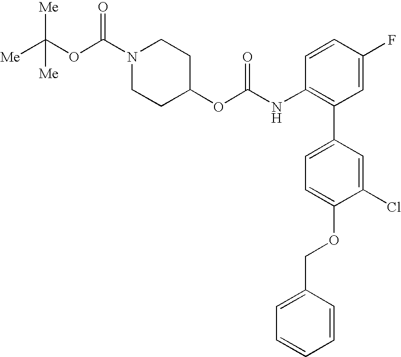

Tert-Butyl 4-({[(2-bromo-4-fluorophenyl)amino]carbonyl}oxy)piperidine-1-carboxylate (1.25 g, 2.99 mmol) (preparation 11), (4-benzyloxy-3-chlorophenyl)boronic acid (1 g, 4.19 mmol), palladium(0), tetrakis(triphenylphosphine) (0.346 g, 0.3 mmol), sodium carbonate (0.889 g, 8.39 mmol), dimethylformamide (15 mL) and water (4 mL) were combined and heated to 105° C. for 5 hours. Diethyl ether (150 mL) was added to the reaction mixture and washed with water (30 mL). Organics were separated and the aqueous layer was washed with diethyl ether (2×150 mL). Organics were combined, dried (sodium sulfate) and concentrated in vacuo to yield a green coloured oil. The oil was purified by column chromatography on silica gel eluting with ethyl acetate:heptane (10/90 by volume) to ethyl acetate:heptane (30/70 by volume) to furnish the title compound as a beige coloured foam, 0.9 g.

¹HNMR (400 MHz, CD₃OD) δ=1.42 (2H, m), 1.44 (9H, s), 3.54 (2H, m), 3.30 (2H, m), 3.67 (2H, m), 4.72 (1H, m), 5.23 (2H, s), 7.07 (2H, m), 7.17 (1H, m), 7.24 (1H, m), 7.31 (1H, m), 7.38 (3H, m), 7.43 (1H, m), 7.48 (2H, m) ppm.

### PREPARATION 13

### piperidin-4-yl[4′-(benzyloxy)-3′-chloro-5-fluorobiphenyl-2-yl]carbamate

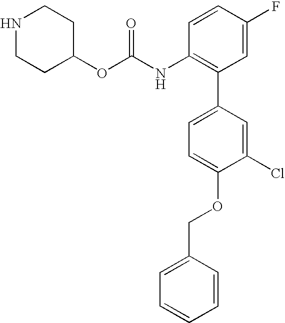

tert-butyl4-[({[4′-(benzyloxy)-3′-chloro-5-fluorobiphenyl-2-yl]amino}carbonyl)oxy]piperidine-1-carboxylate (0.9 g, 1.621 mmol) (preparation 12) and 4.0M hydrogen chloride solution in 1,4-dioxane (10 mL) were combined and stirred at ambient temperature, under nitrogen for 2 hours. The solvent was removed in vacuo. Saturated sodium hydrogen carbonate solution (20 mL) was added with caution. Product was extracted into ethyl acetate (2×30 mL), dried (sodium sulfate) and concentrated in vacuo to yield the title compound as a yellow coloured oil, 0.801 g.

LRMS (ESI): m/z 455 [M+H]⁺

### PREPARATION 14

### di-tert-butyl (9-[4-[({[4′-(benzyloxy)-3′-chloro-5-fluorobiphenyl-2-yl]amino}carbonyl)oxy]piperidin-1-yl]nonyl)imidodicarbonate

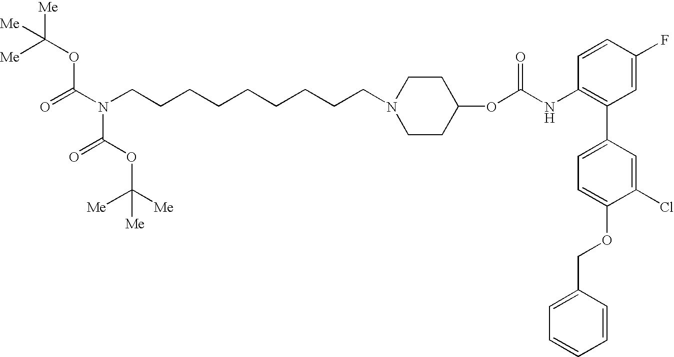

Piperidin-4-yl[4′-(benzyloxy)-3′-chloro-5-fluorobiphenyl-2-yl]carbamate (0.801 g, 1.761 mmol) (preparation 13), (9-bromo-nonyl)dicarbamic acid tert-butyl ester 0.744 g, 1.761 mmol) (preparation 1) and sodium hydrogen carbonate (0.444 g, 5.28 mmol) were suspended in acetonitrile (25 mL). The reaction mixture was heated to 75° C. under nitrogen for 9 hours. The reaction mixture was concentrated in vacuo then dissolved in ethyl acetate (30 mL) and washed with water (20 mL). Ethyl acetate layer was dried (sodium sulphate) and concentrated in vacuo to yield a yellow coloured oil. This oil was purified by column chromatography on silica gel eluting with dichloromethane:methanol:880 ammonia (98/2/0.2 to 96/4/0.4 by volume) to yield the title compound as a white foam, 0.706 g.

LRMS (APCI): m/z 796 [M+H]⁺, 818 [M+Na]⁺.

### PREPARATION 15

### 1-(9-aminononyl)piperidin-4-yl[4′-(benzyloxy)-3′-chloro-5-fluorobiphenyl-2-yl]carbamate

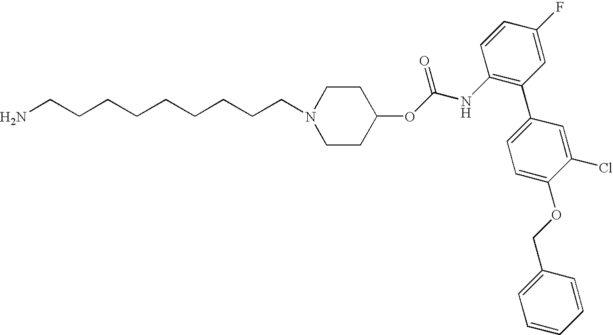

di-tert-Butyl(9-{4-[({[4′-(benzyloxy)-3′-chloro-5-fluorobiphenyl-2-yl]amino}carbonyl)oxy]piperidin-1-yl}nonyl)imidodicarbonate (0.706 g, 0.8865 mmol) (preparation 14) and 4.0M hydrogen chloride solution in 1,4-dioxane (10 mL) were combined and stirred at room temperature under nitrogen for 1.5 hours. The solvent was removed in vacuo. Saturated sodium hydrogen carbonate solution (20 mL) was added with caution. Product was extracted into ethyl acetate (2×30 mL), dried (sodium sulphate) and concentrated in vacuo to yield the title compound as a beige coloured solid, 0.520 g.

LRMS (ESI): m/z 596 [M+H]⁺

### PREPARATION 16

### (1-(9-{[(2R)-2-{4-(benzyloxy)-3-[(methylsulfonyl)amino]phenyl}-2-{[tert-butyl(dimethyl)silyl]oxy}ethyl]amino}nonyl)piperidin-4-yl[4′-(benzyloxy)-3′-chloro-5-fluorobiphenyl-2-yl]carbamate

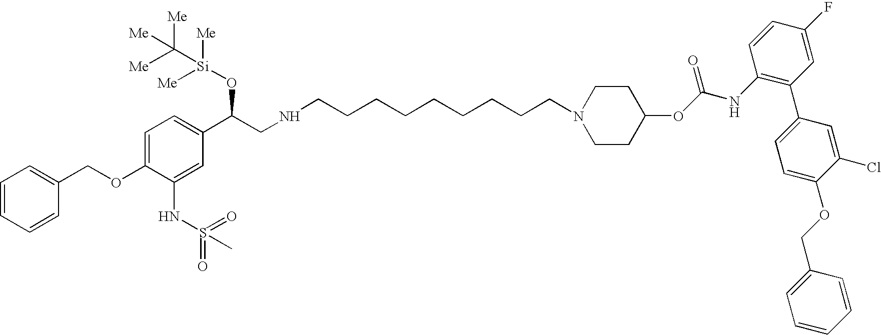

1-(9-aminononyl)piperidin-4-yl[4′-(benzyloxy)-3′-chloro-5-fluorobiphenyl-2-yl]carbamate (0.52 g, 0.872 mmol) (preparation 15), N-{2-(benzyloxy)-5-[(1R)-2-bromo-1-{[tert-butyl(dimethyl)silyl]oxy}ethyl]phenyl}methanesulfonamide (WO2005/080324, 0.449 g, 0.872 mmol), sodium hydrogen carbonate (0.22 g, 2.62 mmol) and acetonitrile (7.0 mL) were combined and heated at 85° C. for 48 hours. Solvent was removed in vacuo leaving a yellow oil which was dissolved in ethyl acetate (30 mL) and washed with water (30 mL). Organics were separated and the aqueous layer was washed with ethyl acetate (30 mL). Organics were combined, dried (sodium sulphate) and concentrated in vacuo to yield a yellow coloured oil. This oil was purified by column chromatography on silica gel eluting with dichloromethane:methanol:880 ammonia (98/2/0.2 to 96/4/0.4 by volume) to yield the title compound as a yellow coloured oil, 0.4 g.

LRMS (ESI): m/z 1030 [M+H]⁺

### PREPARATION 17

### 1-(9-{[(2R)-2-{[tert-butyl(dimethyl)silyl]oxy}-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate

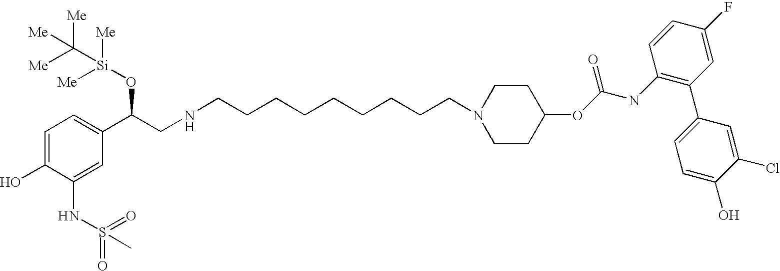

(1-(9-{[(2R)-2-{4-(benzyloxy)-3-[(methylsulfonyl)amino]phenyl}-2-{[tert-butyl(dimethyl)silyl]oxy}ethyl]amino}nonyl)piperidin-4-yl[4′-(benzyloxy)-3′-chloro-5-fluorobiphenyl-2-yl]carbamate (0.4 g, 3.88 mmol) (preparation 16), was dissolved in tert-buty methyl ether (30 mL). 10% Palladium on carbon (0.06 g) was added and the reaction mixture was subjected to hydrogenation conditions at 40° C., 40 psi for 3 hours. The reaction was filtered through Arbocel™ and the filtrate isolated, the solvent was removed in vacuo. The residue was purified by column chromatography on silica gel eluting with dichloromethane:methanol:880 ammonia (98/2/0.2 to 95/5/0.5 by volume) to furnish the title compound, 0.222 g.

LRMS (ESI): m/z 849 [M+H]⁺

### EXAMPLE 1

### 1-(9-{[(2R)-2-Hydroxy-2-{4-hydroxy-3-[(methylsulfonyl)amino]phenyl}ethyl]amino}nonyl)piperidin-4-yl(3′-fluoro-4′-hydroxybiphenyl-2-yl)carbamate

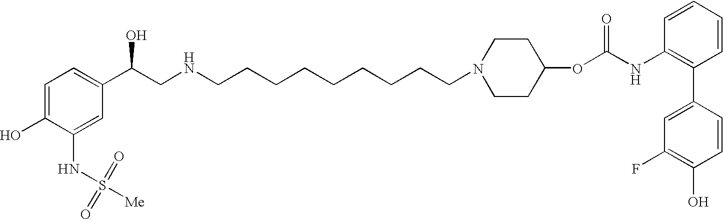

1-(9-{[(2R)-2-{[tert-Butyl(dimethyl)silyl]oxy}-2-{4-hydroxy-3-[(methylsulfonyl)amino]phenyl}ethyl]amino}nonyl)piperidin-4-yl(3′-fluoro-4′-hydroxybiphenyl-2-yl)carbamate (Preparation 8, 264 mg, 0.32 mmol) was dissolved in tetrahydrofuran (5 ml) and triethylaminetrihydrofluoride (261 mg, 1.62 mmol) added in one portion. The reaction was stirred at room temperature for 12 hours and further tetrahydrofuran (6 ml) and 880 ammonia (6 ml) added. The reaction was stirred for 20 minutes and the solvent removed in vacuo, methanol (10 ml) was added and the solvent removed in vacuo. The residue was purified by column chromatography on silica gel eluting with dichloromethane:methanol:880 ammonia (98/2/0.2 to 87/13/1.3 by volume) to furnish the title compound as a brown solid, 106 mg.

LRMS (ES): m/z 701 [M+H]⁺.

### EXAMPLE 2

### 1-(9-{[(2R)-2-Hydroxy-2-{4-hydroxy-3-[(methylsulfonyl)amino]phenyl}ethyl]amino}nonyl)piperidin-4-yl(3′-chloro-4′-hydroxybiphenyl-2-yl)carbamate

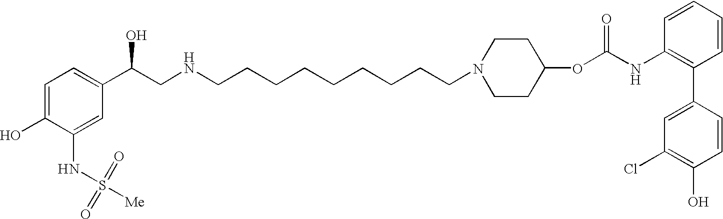

1-(9-{[(2R)-2-{[tert-Butyl(dimethyl)silyl]oxy}-2-{4-hydroxy-3-[(methylsulfonyl)amino]phenyl}ethyl]amino}nonyl)piperidin-4-yl(3′-chloro-4′-hydroxybiphenyl-2-yl)carbamate (Preparation 10, 1.08 g, 1.30 mmol) was dissolved in methanol (30 ml) and triethylaminetrihydrofluoride (230 mg, 1.43 mmol) added in one portion. The reaction was stirred at room temperature for 12 hours and further triethylaminetrihydrofluoride (230 mg, 1.43 mmol) added and the reaction stirred at room temperature for 12 hours. The solvent was removed in vacuo and the residue was purified by column chromatography on silica gel eluting with dichloromethane:methanol:880 ammonia (95/5/0.5 by volume) to furnish the title compound as a white foam, 200 mg.

LRMS (ES): m/z 717,719 [M+H]⁺.

### EXAMPLE 3

### 1-(9-{[(2R)-2-hydroxy-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate

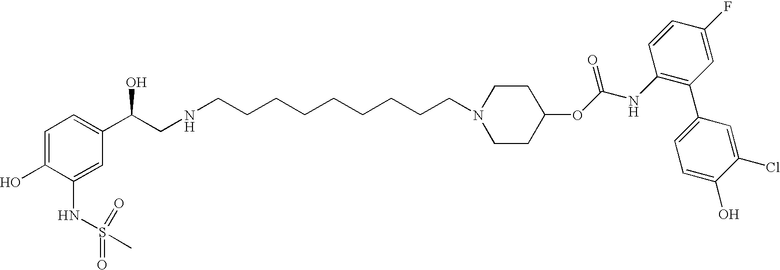

1-(9-{[(2R)-2-{[tert-butyl(dimethyl)silyl]oxy}-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate (0.222 g, 0.261 mmol) (preparation 17) was dissolved in tetrahydrofuran (4 mL). Triethylamine tris-hydrofluoride (0.213 mL, 1.31 mmol) was added and the reaction mixture was stirred at room temperature for 4 hours. 880 Ammonia (0.1 mL) was added, then the reaction mixture was diluted with dichloromethane (30 mL) and washed with saturated sodium hydrogen carbonate solution. Organics were separated and the aqueous was washed with dichloromethane (30 mL). Organics were combined, dried (sodium sulphate) and concentrated in vacuo to yield a beige coloured solid. This solid was purified by column chromatography on silica eluting with dichloromethane:methanol:880 ammonia (95/5/0.5 to 80/20/2 by volume) to yield the title compound as a white solid, 0.095 g.

LRMS (ESI): 735 [M+H]⁺

### EXAMPLE 4

### 1-(9-{[(2R)-2-hydroxy-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate.naphthalene 1,5-disulphonate

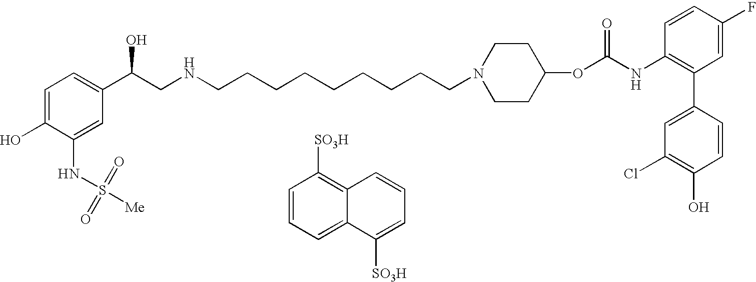

1-(9-{[(2R)-2-hydroxy-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate (0.027 g, 0.0367 mmol) (example 3) was dissolved in methanol (10 mL). A solution of 1,5-naphthalenedisulphonic acid tetrahydrate (0.0132 g, 0.0367 mmol) in methanol (1 mL) was added and the solution was stored at ambient temperature for 65 hours. Title product (white crystalline solid) was filtered from mixture and dried under vacuum, 0.004 g.

¹HNMR (400 MHz, CD₃OD) δ=2.94 (3H, s—diagnostic peak for NHSO₂Me), 9.01 (2H, d—diagnostic peak for naphthalene protons) ppm.

### EXAMPLE 5

### 1-(9-{[(2R)-2-Hydroxy-2-{4-hydroxy-3-[(methylsulfonyl)amino]phenyl}ethyl]amino}nonyl)piperidin-4-yl(3′-chloro-4′-hydroxybiphenyl-2-yl)carbamate naphthalene 1,5-disulphonate

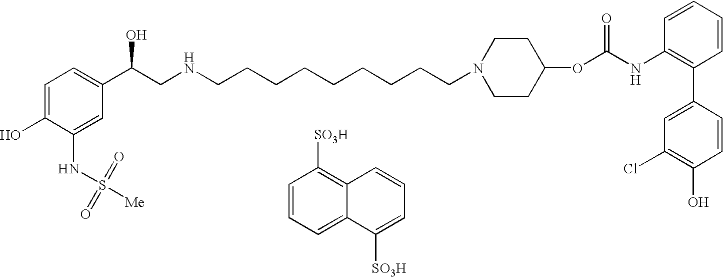

1-(9-{[(2R)-2-Hydroxy-2-{4-hydroxy-3-[(methylsulfonyl)amino]phenyl}ethyl]amino}nonyl)piperidin-4-yl(3-‘chloro-4’-hydroxybiphenyl-2-yl)carbamate (120 mg, 0.17 mmol) (example 2) was dissolved in methanol (4 mL). A solution of 1,5-naphthalenedisulphonic acid tetrahydrate (60 mg, 0.17 mmol) in methanol (2 mL) was added and the solution was stored at ambient temperature until a white precipitate formed (5 hours). The mixture was filtered, washed with cold methanol and dried under vacuum to provide the title compound (68 mg) as a white crystalline solid.

¹HNMR (400 MHz, CD₃OD) δ=2.93 (3H, s—diagnostic peak for NHSO₂Me), 9.01 (2H, d—diagnostic peak for naphthalene protons) ppm.

### EXAMPLE 6

### 1-(9-{[(2R)-2-Hydroxy-2-{4-hydroxy-3-[(methylsulfonyl)amino]phenyl}ethyl]amino}nonyl)piperidin 4-yl(3-‘fluoro-4’-hydroxybiphenyl-2-yl)carbamate naphthalene 1,5-disulphonate

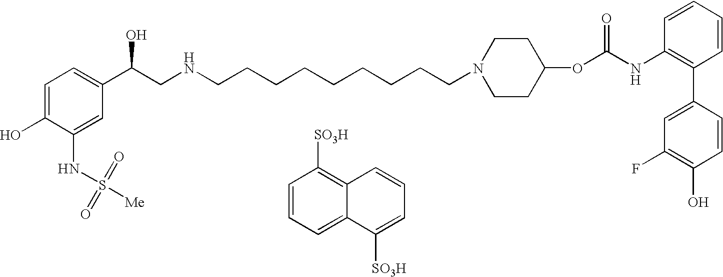

1-(9-{[(2R)-2-Hydroxy-2-{4-hydroxy-3-[(methylsulfonyl)amino]phenyl}ethyl]amino}nonyl)piperidin-4-yl(3-‘fluoro-4’-hydroxybiphenyl-2-yl)carbamate (160 mg, 0.23 mmol) (example 1) was dissolved in methanol (20 mL). A solution of 1,5-naphthalenedisulphonic acid tetrahydrate (66 mg, 0.23 mmol) in methanol (5 mL) was added and the solution was left for 96 hours before removal of approximately half the methanol on a rotary evaporator. The mixture was heated to 70° C. for 5 minutes to achieve total dissolution and then allowed to reach room temperature slowly over night. A precipitate formed which was filtered off and dried under vacuum to provide the title compound (40 mg) as a pale brown crystalline solid.

¹HNMR (400 MHz, DMSO-d₆) δ=2.95 (3H, s—diagnostic peak for NHSO₂Me), 8.88 (2H, d—diagnostic peak for naphthalene protons) ppm.

Functional Assessment of Antagonist Activity Using a Whole Cell β-Lactamase Reporter Assay in CHO Cells Expressing the hM₃ Receptor.

Cell Culture

CHO (Chinese Hamster Ovary) cells recombinantly expressing the human muscarinic M₃ receptor were transfected with the NFAT_β-Lac_Zeo plasmid. Cells were grown in DMEM with Glutamax-1, supplemented with 25 mM HEPES (Life Technologies 32430-027), containing 10% FCS (Foetal Calf Serum; Sigma F-7524), 1 nM Sodium pyruvate (Sigma S-8636), NEAA (non-Essential Amino Acids; Invitrogen 11140-035) and 200 μg/ml Zeocin (Invitrogen R250-01).

hM3 β-Lac Assay Protocol

Cells were harvested for assay when they reached 80-90% confluency using enzyme free cell Dissociation Solution (Life technologies 13151-014) incubated with the cells for 5 min at 37° C. in an atmosphere containing 5% CO₂. Detached cells were collected in warmed growth media and centrifuged at 2000 rpm for 10 min, washed in PBS (Phosphate Buffered Saline; Life Technologies 14190-094) and centrifuged again as just described. The cells were re-suspended at 2×10⁵ cells/ml in growth medium (composition as described above). 20 μl of this cell suspension was added to each well of a 384 well black clear bottomed plate (Greiner Bio One 781091-PFI). The assay buffer used was PBS supplemented with 0.05% Pluronic F-127 (Sigma 9003-11-6) and 2.5% DMSO. Muscarinic M₃ receptor signalling was stimulated using 80 nM carbamyl choline (Aldrich N240-9) incubated with the cells for 4 h at 37° C./5% CO₂ and monitored at the end of the incubation period using a Tecan SpectraFluor+ plate reader (λ-excitation 405 nm, emission 450 nm and 503 nm). Compounds under test were added to the assay at the beginning of the 4 h incubation period and compound activity measured as the concentration dependent inhibition of the carbamyl choline induced signal. Inhibition curves were plotted and IC₅₀ values generated using a 4-parameter sigmoid fit and converted to Ki values using the Cheng-Prusoff correction and the K_{D} value for carbamyl choline in the assay.

Functional Assessment of Agonist Potency and Efficacy Using a Whole Cell Luciferase Reporter Assay in CHO Cells Expressing the hB₂ Receptor.

Cell Culture

CHO (Chinese Hamster Ovary) cells recombinantly expressing the human adrenergic B₂ receptor and transfected with a luciferase enzyme reporter gene were maintained in growth media composed of F12:DMEM (Sigma D6421) containing 10% Foetal Bovine Serum (FBS: Sigma F03921) 10 μg/ml puromycin (Sigma N277698), 0.5 mg/ml Geneticin G418 (Sigma G7034) and 2 mM L-glutamine (Sigma G7513). The cells were kept in sterile conditions at 37° C., in an atmosphere containing 5% CO₂.

hB2 Luciferase Assay Protocol

Cells were harvested for assay when they reached 80-90% confluency using enzyme free cell Dissociation Solution (Life technologies 13151-014) incubated with the cells for 5 min at 37° C. in an atmosphere containing 5% CO₂. Detached cells were collected in warmed growth media (composition described above), and re-suspended in assay media (F12:DMEM (Sigma D6421) containing 1% Foetal Bovine Serum (FBS: Sigma F03921), 10 μg/ml puromycin (Sigma N277698), 0.5 mg/ml Geneticin G418 (Sigma G7034) and 2 mM L-glutamine (Sigma G7513)) to give a viable cell concentration of 1×106 cells/ml. 10 ul of this suspension was added to each well of a tissue culture treated low volume 384 well plate (Greiner788073) and the plate incubated in an atmosphere containing 5% CO₂ at 37° C. for 2 h. Concentration ranges of test compounds were prepared in phosphate Buffered Saline containing 0.05% pluronic-F127 (Sigma P2443) and 2.5% DMSO. 2 μl of each test concentration were added to the appropriate 384 plate well and returned to the incubator for a further 4 h. At the end of the incubation period 4 μl of Steady-Glo reagent (Steady-Glo Luciferase assay system (Promega E2520) was added to each well and the plate read immediately in a Leadseeker Plate reader (Amersham Bioscience) using a 660 nm filter. Concentration effect curves were plotted and EC₅₀ values generated using a 4-parameter sigmoid fit using an in-house data analysis programme. Isoprenaline was run in every assay as a reference standard.

Examples 1 and 2 were tested according to the above-disclosed assays and the following results were obtained:

| Example | EC₅₀ - beta2 (nM) | IC₅₀ - M3 (nM) |
|---|---|---|
| 1 | 1.01 (n = 2) | 2.93 (n = 2) |
| 2 | 0.133 (n = 3) | 0.725 (n = 6) |
| 3 | 0.252 (n = 5) | 1.07 (n = 3) |

1. A compound of formula (1), 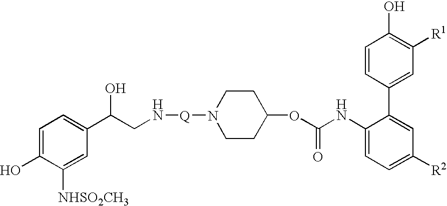
wherein
R¹ is halo;
R² is H or halo; and
Q is —(CH₂)₉— or 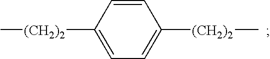
or a pharmaceutically acceptable salt or solvate thereof.

2. A compound of claim 1, or a pharmaceutically acceptable salt or solvate thereof, wherein R¹ is F.

3. A compound of claim 1, or a pharmaceutically acceptable salt or solvate thereof, wherein R¹ is Cl.

4. A compound of claim 1, or a pharmaceutically acceptable salt or solvate thereof, wherein Q is —(CH₂)₉—.

5. A compound of claim 1, or a pharmaceutically acceptable salt or solvate thereof, wherein Q is

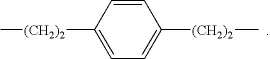

6. A compound of claim 1, or a pharmaceutically acceptable salt or solvate thereof, wherein R² is H.

7. A compound of any claim 1, or a pharmaceutically acceptable salt or solvate thereof, wherein R² is H.

8. The R-stereoisomer of a compound of claim 1, or a pharmaceutically acceptable salt or solvate thereof.

9. A compound of claim 1 selected from the group consisting of
1-(9-{[(2R)-2-Hydroxy-2-{4-hydroxy-3-[(methylsulfonyl)amino]phenyl}ethyl]amino}nonyl)piperidin-4-yl(3′-fluoro-4′-hydroxybiphenyl-2-yl)carbamate;
1-(9-{[(2R)-2-Hydroxy-2-{4-hydroxy-3-[(methylsulfonyl)amino]phenyl}ethyl]amino}nonyl)piperidin-4-yl(3′-chloro-4′-hydroxybiphenyl-2-yl)carbamate; and
1-(9-{[(2R)-2-hydroxy-2-{4-hydroxy-3-[(methylsulfonyl)amino]phenyl}ethyl]amino}nonyl)piperidin-4-yl(3′-chloro-5-fluoro-4′-hydroxybiphenyl-2-yl)carbamate; or
a pharmaceutically acceptable salt or solvate thereof.

10. A pharmaceutical composition comprising a a compound of claim 1 or a pharmaceutically acceptable salt or solvate thereof.

11. A pharmaceutical composition of claim 10, further comprising one or more pharmaceutically acceptable excipients and/or additives.

12. A method of treating asthma, bronchitis, obstructive or inflammatory airways disease, acute lung injury or bronchiectasis in a mammal, said method comprising administering to said mammal a therapeutically effective amount of a compound of claim 1 or a pharmaceutically acceptable salt or solvate thereof, or a pharmaceutical composition comprising a compound of claim 1 or a pharmaceutically acceptable salt thereof and a pharmaceutically acceptable excipient or additive.

13. A method of claim 12 wherein said asthma is selected from atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

14. A method of claim 12 wherein said bronchitis is selected from acute bronchitis, chronic bronchitis, acute laryngotracheal bronchitis, arachidic bronchitis, catarrhal bronchitis, croupus bronchitis, dry bronchitis, infectious asthmatic bronchitis, productive bronchitis, staphylococcus bronchitis, streptococcal bronchitis and vesicular bronchitis.

15. A method of claim 12 wherein said obstructive or inflammatory airways disease is selected from chronic eosinophilic pneumonia, chronic obstructive pulmonary disease (COPD), COPD that includes chronic bronchitis, pulmonary emphysema or dyspnea associated or not associated with COPD, COPD that is characterized by irreversible, progressive airways obstruction, adult respiratory distress syndrome (ARDS), exacerbation of airways hyper-reactivity consequent to other drug therapy and airways disease that is associated with pulmonary hypertension.

16. A method of claim 12 wherein asthma or chronic obstructive pulmonary disease is treated.

17. A pharmaceutical composition comprising a combination of a compound of claim 1, or a pharmaceutically acceptable salt or solvate thereof, with a therapeutic agent selected from:
(a) a 5-Lipoxygenase (5-LO) inhibitor or 5-lipoxygenase activating protein (FLAP) antagonist;
(b) an antagonist of LTB₄, LTC₄, LTD₄, or LTE₄;
(c) an antagonist of H1 or H3;
(d) α₁- or α₂-adrenoceptor agonist vasoconstrictor sympathomimetic agent;
(e) a PDE3, PDE4 or PDE5 inhibitor;
(f) Theophylline;
(g) Sodium cromoglycate;
(h) a non-selective or selective COX-1 or COX-2 inhibitor;
(i) a prostaglandin receptor antagonist or inhibitor of prostaglandin synthase;
(j) an oral or inhaled glucocorticosteroid;
(k) a monoclonal antibody active against endogenous inflammatory entities;
(l) an Anti-tumor necrosis factor agent;
(m) an adhesion molecule inhibitor;
(n) a kinin-B₁- or B₂-receptor antagonist;
(o) an immunosuppressive agent;
(p) an inhibitor of matrix metalloprotease (MMP);
(q) a tachykinin NK₁, NK₂ or NK₃ receptor antagonists,
(r) an elastase inhibitor;
(s) an adenosine A2a receptor agonist;
(t) an inhibitor of urokinase;
(u) a D2 agonist;
(v) a modulator of the NFκβ pathway;
(w) a p38 MAP kinase, syk kinase or JAK kinase inhibitor;
(x) a mucolytic or anti-tussive agent;
(y) an agent which enhances response to inhaled corticosteroids;
(z) an antibiotic or antiviral agent effective against micro-organisms which can colonise the respiratory tract;
(aa) a DP1, DP2 or CRTH2 antagonist,
(bb) an HDAC inhibitor;
(cc) a PI3 kinase inhibitor;
(dd) a p38 inhibitor; or
(ee) a CXCR2 antagonist.

18. A compound of formula (12) 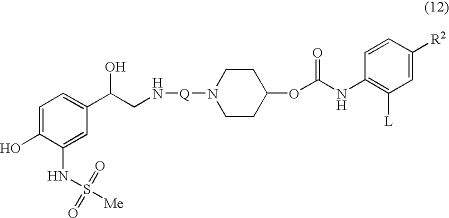
wherein L is a leaving group and wherein
R² is H or halo; and
Q is —(CH₂)₉— or

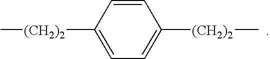

19. A compound of formula (13) 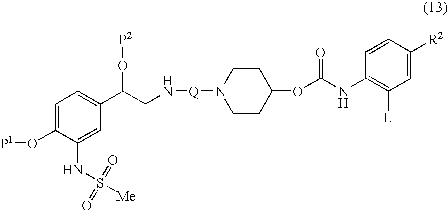
wherein R² is H or halo;
Q is —(CH₂)₉— or 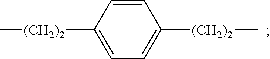
L is a leaving group; and
P¹ and P² are suitable hydroxyl protecting groups.
